(19) United States
(12) Patent Application Publication
(10) Pub. No.: US 2003/0082104 A1
(43) Pub. Date: May 1, 2003

(54) Imaging method and imaging apparatus, in particular for small animal imaging

(76) Inventor: Thomas Mertelmeier, Erlangen (DE)

Correspondence Address: YOUNG & THOMPSON, 745 SOUTH 23RD STREET 2ND FLOOR, ARLINGTON, VA, 22202

(21) Appl. No.: 10/273,994

(22) Filed: Oct. 21, 2002

(30) Foreign Application Priority Data
Oct. 19, 2001 (DE) 10151670.3

(51) Int. Cl. A61K 49/04; G21K 1/12; H05G 1/60

(52) U.S. Cl. 424/9.4; 424/9.6; 378/21

(57) ABSTRACT

An imaging method and apparatus, for small animal imaging, in which the object to be examined is treated with an activatable optical contrast medium, which preferably has a metabolically activatable mark, and irradiated by a first optical excitation source. The first radiation reflected from the object is detected by a first optical detector, and simultaneously irradiated by a second tomographic excitation source, while the second radiation transferred from the object is detected by a second tomographic detector. In this case, the second tomographic excitation source advantageously generates an X-ray radiation, so that the resulting CT image can be superposed with the optical image in order to evaluate morphological and functional information.

### FIELD OF THE INVENTION

The invention relates to an imaging method and to an imaging apparatus, in particular for small animal imaging.

### BACKGROUND OF THE INVENTION

Known imaging methods and apparatuses for small animal imaging comprise a first optical excitation source, which irradiates an object treated with an activatable optical contrast medium, such as, for example, a mouse or a rat, while the radiation reflected from the object is detected by an optical detector. In examinations of metabolic functions on a living small animal, use is made of activatable optical contrast media which fluoresce in particular in the near infrared region. The contrast medium is inert in healthy tissue and is activated, that is to say transferred into a fluorescent state, only in the target tissue to be detected, for example a tumor, by illness-correlated metabolic activities (enzymatic processes). Through a highly selective activation mechanism, a very high signal-to-noise ratio is achieved with these contrast media. For this purpose, the contrast media have metabolic markers which react to specific metabolic functions and activate the contrast medium. Essentially functional information of the target region can thereby be detected.

Such imaging methods and apparatuses are disclosed for example in DE 195 23 806 A1 and DE 198 04 797 A1. The latter show an illumination system with an optical light source which emits excitation light which is adapted to the fluorescence excitation spectrum of the tissue to be examined. The intensities of the reflected radiation are detected by an optical detector and evaluated. The latter also detects the fluorescence radiation of the regions of interest. DE 195 23 806 A1 furthermore discloses a second optical light source which partially runs in the beam path of the first light radiation to the surface having fluorescent substances and generates an image—stationary for the observer—of the distribution of fluorescent substances on the surface, if the first light beam forms a sufficiently fast surface scanning movement. DE 198 04 797 A1 also discloses the use of a second optical light source, which illuminates the object field or the surface for visual observation.

This optical imaging modality has the disadvantage, however, that the spatial resolution of the reflected radiation is greatly restricted on account of the high degree of scattering and the absorption of light in the target tissue. It is thus virtually impossible to detect any anatomical and/or morphological information of the examination site.

Another imaging modality is micro-CT (computer tomography). The latter yields anatomical and/or morphological information with high spatial resolution, since corresponding X-ray radiation is absorbed by the tissue to be examined and the transferred radiation thereby mirrors anatomical conditions with high accuracy. On the other hand, owing to the relatively low X-ray absorption, micro-CT is insensitive to metabolism-specific contrast media used for example for nuclear medicine.

Furthermore, imaging methods are known according to which firstly anatomical and morphological information is determined by means of a radiograph in order then to determine functional sectional images with the aid of optical imaging methods, which images are then evaluated with the aid of the X-ray images. These methods have the disadvantage, however, that as a general rule it is not possible to unambiguously assign the functional information to the anatomical information and accurate evaluation of the image information has therefore been possible only with the aid of appropriate experience.

### SUMMARY OF THE INVENTION

Therefore, the present invention is based on the object of improving an imaging method and an imaging apparatus of conventional design in such a way that it is possible to detect both anatomical information with high spatial resolution and functional information with high sensitivity from a target tissue.

According to the invention, in the case of an imaging method which is suitable in particular for small animal imaging, the object to be examined is treated with an activatable optical contrast medium and irradiated by a first optical excitation source, the first radiation reflected from the object being detected by a first optical detector. Furthermore, the object to be examined is simultaneously irradiated by a second excitation source, the second radiation of the second excitation source which is transferred from the object being detected by a second detector. The optical imaging system is thus advantageously combined with the tomographic imaging system, without the object having to be displaced. Consequently, different items of information of the same target tissue are determined simultaneously, which items of information are not only evaluated individually in each case but, on account of the fact that both imaging methods are carried out simultaneously, can also be correlated with one another.

Advantageously, the object to be examined is firstly treated with an optical fluorescence contrast medium which has at least one metabolically activatable marker, so that fluorescent radiation that is radiated back can be detected by means of the first detector. The fluorescent radiation that is radiated back and detected is reconstructed, thereby producing a corresponding image with functional information. In this case, the reconstruction for optical tomography is advantageously carried out iteratively (e.g. R. Gaudette et al.: Phys. Med. Biol. 45, 1051-1070 (2000), A. D. Klose, A. H. Hielscher: Med. Phys. 26, 1698-1707 (1999), H. Dehghani, D. T. Delpy, S. R. Arridge: Phys. Med. Biol. 44, 2897-2906 (1999), S. A. Arridge, J. C. Hebden, Phys. Med. Biol. 42, 841-853 (1997)).

The second excitation source is advantageously an X-ray tube which generates an X-ray radiation. This X-ray radiation transilluminates the object and is detected by the second detector which is designed as a CT detector, for example. The X-ray image thereby determined contains corresponding anatomical and morphological information with high spatial resolution. However, the second excitation source could also be an ultrasonic transducer or a magnetic resonance tomograph.

The X-ray attenuation coefficients which can be measured by the radiograph can advantageously be used as prior information. The initial concentration of the contrast medium can advantageously be determined by means of the attenuation coefficient of the second X-ray radiation transferred from the object, which initial concentration can be used for example for quantifying the metabolic activity by determining the activation rate.

Furthermore, by way of example, the X-ray attenuation coefficient advantageously serves for determining optical scattering and/or absorption coefficients for the evaluation of the first reflected optical radiation. The first optical imaging preferably involves fluorescence in the near infrared region (NIRF), for which new intelligent fluorescence contrast media have been developed (cf. R. Weissleder et al.: Nature Biotechnology 17, 375-368 (1999)). Consequently, an absorption and scattering coefficient can be estimated from each voxel determined by means of the optical imaging method. The fluorescence activity can therefore be determined qualitatively and quantitatively more exactly.

The reflected first radiation detected by the first detector is advantageously evaluated and converted into corresponding functional image information. The transferred second radiation detected by the second detector is likewise evaluated, thereby producing an image data record with morphological image information. The resultant individual image information items can then be superposed to form a total image with morphological and functional information of the same target tissue.

According to the invention, it is also possible for a plurality of sectional images of the object to be supplemented to form a three-dimensional image data record. For this purpose, the transferred second radiations which are detected by the second detector and represent a plurality of sections of the object are evaluated and used to generate the three-dimensional image data record. Missing image data between the individual two-dimensional sectional images are interpolated or estimated according to known methods (volume rendering). Afterward, the three-dimensional image information can be supplemented or superposed by means of the functional image information of the first detector in order to obtain a three-dimensional image data record which also contains functional image information. For accurate assignment of the different image information items and in order to avoid position artifacts it is possible to use anatomical and/or artificial landmarks. For this purpose, use is made, for example, of small light-emitting diodes on the surface of an object carrier or corresponding small metal balls, which are visible in the CT image.

Moreover, the optical function information can be superposed from a planar optical image into the X-ray projection image, which has been recorded with the same projection angle.

The invention therefore has various advantages over the conventional systems. Both anatomical and functional information can be detected with one device. The system can be set up in decentralized fashion and has small dimensions. Furthermore, it is more cost-effective than other “single modality” systems, such as PET or MRI, for example. The optical reconstruction can be reliably improved by means of the CT information. Anatomical information with high spatial resolution and functional information with high sensitivity are therefore provided simultaneously.

### BRIEF DESCRIPTION OF THE DRAWINGS

A preferred embodiment of the present invention is explained below with reference to the drawings, in which:

FIG. 1 shows a diagrammatic illustration of the imaging apparatus according to the invention,

FIG. 2 shows a diagrammatic illustration of an alternative imaging apparatus, and

FIG. 3 shows a second alternative embodiment of the imaging apparatus according to the invention.

### DETAILED DESCRIPTION OF THE INVENTION

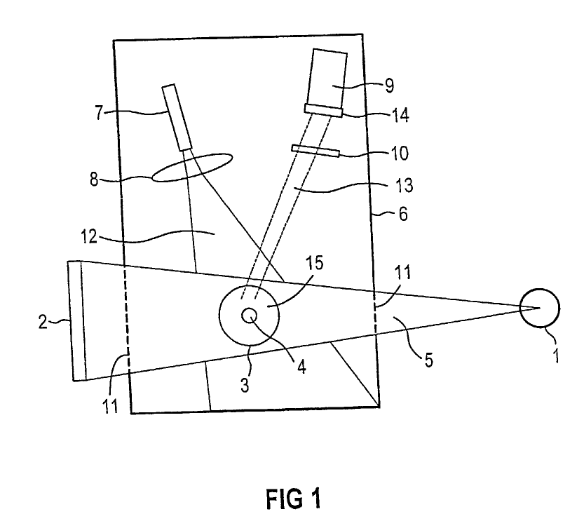

FIG. 1 diagrammatically shows an imaging apparatus according to the invention having a first, optical excitation source 7, which irradiates an object 15 to be examined, which has been treated with an activatable optical contrast medium, and a first optical detector 9, which detects first radiation 13 reflected from the object 15. A second excitation source 1 simultaneously irradiates the object 15 to be examined. A second detector 2 detects the second radiation 5 transferred from the object 15.

Advantageously, the second excitation source 1 is an X-ray tube and the second detector 2 is an X-ray detector. An object carrier 3, which holds the object 15, is advantageously a cylinder made of glass or Plexiglas which can be pivoted or rotated about an axis 4 of rotation. This object carrier 3 is transmissive both for X-ray radiation and for light. While the object 15 is mounted rotatably (turntable) on the object carrier 3, both the X-ray system (1, 2) and the optical recording system (7, 9) are arranged in a stationary manner. The optical recording system is advantageously located within a housing 6 which is opaque, i.e. light-tight.

The second radiation 5, i.e. for example the X-ray cone beam which is radiated by the X-ray tube 1, passes through an X-ray-transparent window 11 in the light-tight housing 6 on both sides of the object 15 and is detected by the second detector 2.

The first excitation source 7 is advantageously an infrared laser diode which radiates infrared light via a lens 8 onto the object 15. The first incident radiation 12, for example infrared light, excites the optical contrast medium present in the object, thereby producing a reflected first radiation 13, i.e. for example reflected fluorescent light. This reflected radiation 13 is detected, via a filter 10 and a lens 14, by the first detector 9, for example a CCD camera.

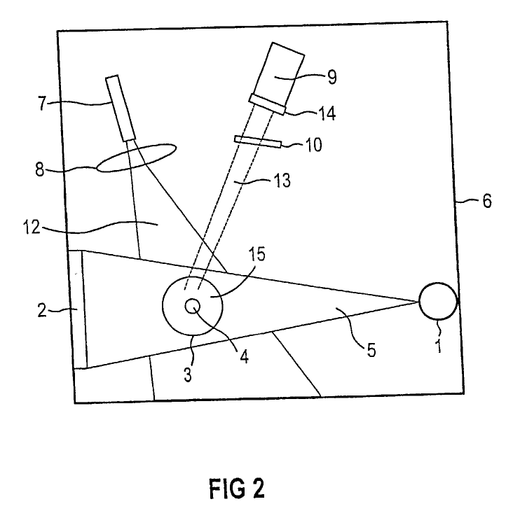

FIG. 2 shows an alternative embodiment of the imaging apparatus according to the invention. This differs from the embodiment according to FIG. 1 by the fact that the second excitation source 1 and the second detector 2 are also situated within the light-tight housing 6.

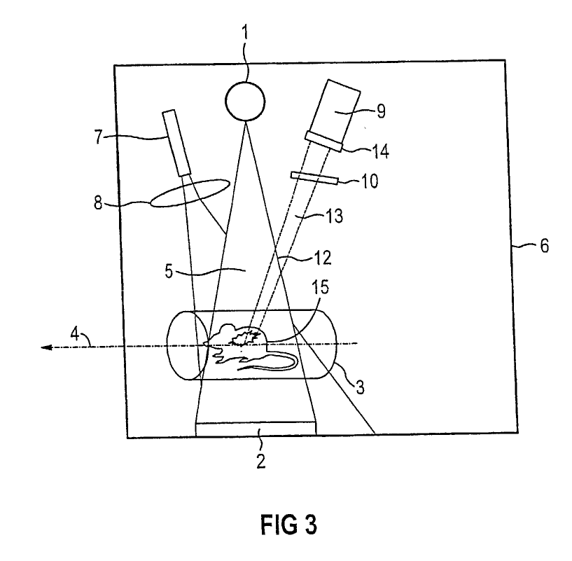

FIG. 3 shows a further alternative embodiment of the imaging apparatus according to the invention. In this case, too, both imaging systems are situated within the light-tight housing 6, while the object 15 is displaced progressively along the axis 4. This makes it possible to record progressively different sectional images of the tissue to be examined, which can then be combined to form a three-dimensional CT image. With the use of an extensive X-ray detector 2, the entire object can be 3-D reconstructed (cone beam tomography). This three-dimensional image can be supplemented with the functional information by means of the optical images that are likewise obtained at the same time.

The reconstructed X-ray attenuation coefficient at a site of the target tissue is advantageously used as an estimated value for the optical absorption and/or scattering coefficient at this site during the iterative optical reconstruction, so that the inverse problem of optical imaging can be alleviated.

1. An imaging method, for small animal imaging, which comprises:
treating the object to be examined with an activatable optical contrast medium;
irradiating said treated object by a first optical excitation source;
detecting the first radiation reflected from the object by a first optical detector;
simultaneously irradiating by a second tomographic excitation source; and
detecting the second radiation transferred from the object by a second tomographic detector.

2. The imaging method as claimed in claim 1, wherein the object to be examined is treated with an optical fluorescence contrast medium which has at least one metabolically activatable marker, so that fluorescent radiation that is radiated back is detected by the first detector.

3. The imaging method as claimed in claim 1, wherein the second tomographic excitation source generates an X-ray radiation, and the second tomographic detector is a CT detector.

4. The imaging method as claimed in claim 1, wherein an attenuation coefficient of the second radiation transferred from the object is used in order to determine the initial concentration of the contrast medium.

5. The imaging method as claimed in claim 1, wherein an attenuation coefficient of the second radiation transferred from the object is used in order to determine optical scattering and/or absorption coefficients for the evaluation of the first radiation.

6. The imaging method as claimed in claim 1, wherein the reflected first radiation detected by the first optical detector is evaluated and converted into functional image information,
the transferred second radiation detected by the second tomographic detector is evaluated and converted into morphological image information, and
the resultant individual image information items are superposed to form a total image with morphological and functional information.

7. The imaging method as claimed in claim 1, wherein transferred second radiations which are detected by the second tomographic detector and represent a plurality of sections of the object are evaluated and used to generate a three-dimensional image data record, and
the functional image information of the first optical detector is superposed with the three-dimensional image data record.

8. The imaging method as claimed in claim 6, wherein anatomical and/or artificial landmarks are used for the superposition of the information.

9. The imaging method as claimed in claim 7, wherein anatomical and/or artificial landmarks are used for the superposition of the information.

10. An imaging apparatus, for small animal imaging comprising:
a first optical excitation source, which irradiates an object to be examined which has been treated with an activatable optical contrast medium;
a first optical detector, which detects first radiation reflected from the object, a second tomographic excitation source which simultaneously irradiates the object to be examined; and
a second tomographic detector which detects the second radiation transferred from the object.

11. The apparatus as claimed in claim 10, wherein the second tomographic excitation source is an X-ray tube, and the second tomographic detector is a CT detector.

12. The apparatus as claimed in claim 10, wherein the first optical excitation source is an infrared laser source, and the first detector is a CCD camera.

13. The apparatus as claimed in claim 10, wherein the object can be mounted on an object carrier made of glass or Plexiglas, said object carrier being rotatable about an axis of rotation.

14. The apparatus as claimed in claim 11, wherein the first optical excitation source is an infrared laser source, and the first detector is a CCD camera.

15. The apparatus as claimed in claim 11, wherein the object can be mounted on an object carrier made of glass or Plexiglas, said object carrier being rotatable about an axis of rotation.

16. The apparatus as claimed in claim 12, wherein the object can be mounted on an object carrier made of glass or Plexiglas, said object carrier being rotatable about an axis of rotation.
